SEQUENCE LISTING (5 sequences)

SEQ ID NO: 1 - protein, 269 aa, Bacillus lentus
  Seq 1 Bacillus lentus alkalische Protease R99E
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus lentus
  Seq 2 Subtilisin 309
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 275 aa, Bacillus amyloliquefaciens [sequence omitted]

SEQ ID NO: 4 - protein, 274 aa, Bacillus lentus
  Seq 4 Subtilisin Carlsberg mature
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 5 - protein, 269 aa, Bacillus lentus
  Seq 5 Bacillus lentus alkalische Protease WT
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2017/0253863 A1
(43) Pub. Date: Sep. 7, 2017

(54) PROTEASES WITH ENHANCED WATER HARDNESS TOLERANCE

(71) Applicant: Henkel AG & Co. KGaA, Dusseldorf (DE)

(72) Inventors: Thomas Weber, Dormagen (DE); Hendrik Hellmuth, Darmstadt (DE); Timothy O'Connell, Landsberg am Lech (DE); Inken Prueser, Duesseldorf (DE)

(73) Assignee: Henkel AG & Co. KGaA, Dusseldorf (DE)

(21) Appl. No.: 15/129,989

(22) PCT Filed: Mar. 11, 2015
(86) PCT No.: PCT/EP2015/055036

§ 371 Date: Sep. 28, 2016

(30) Foreign Application Priority Data
Mar. 31, 2014 (DE) 102014206051.6

(51) Int. Cl. C12N 9/54; C11D 3/386

(52) U.S. Cl. CPC C12N 9/54; C11D 3/386; C12Y 304/21062

(57) ABSTRACT

The invention relates to proteases with enhanced water hardness tolerance and to protease-containing washing or cleaning agents.

### CROSS-REFERENCE TO RELATED APPLICATIONS

This application is a U.S. National-Stage entry under 35 U.S.C. §371 based on International Application No. PCT/EP2015/055036, filed Mar. 11, 2015, which was published under PCT Article 21(2) and which claims priority to German Application No. 10 2014 206 051.6, filed Mar. 31, 2014, which are all hereby incorporated in their entirety by reference.

### FIELD OF THE INVENTION

The present invention generally relates to proteases with enhanced water hardness tolerance and to protease-containing washing or cleaning agents.

### BACKGROUND OF THE INVENTION

Washing or cleaning agents, particularly washing or cleaning agents for washing dishes by machine and for cleaning textiles, generally contain one or more enzymes as further cleaning-active ingredients in addition to the builders and surfactants.

Proteases, and thereunder in particular serine proteases, which also include the subtilases, are the longest-established enzymes and are contained in practically all modern, effective washing or cleaning agents. They cause the breakdown of protein-containing stains on the items to be cleaned. Hereunder, in turn, proteases of the subtilisin type (subtilases, subtilopeptidases, EC 3.4.21.62) are especially important, which are classed as serine proteases because of the catalytically active amino acids. They act as non-specific endopeptidases, i.e., they hydrolyze any acid-amide bonds located in the interior of peptides or proteins. Their pH optimum is usually in the clearly alkaline range. An overview of this family is provided, for example, by the article “Subtilases: Subtilisin-like Proteases” by R. Siezen, pages 75-95 in “Subtilisin enzymes,” published by R. Bott and C. Betzel, New York, 1996. Of course, subtilases are formed by microorganisms; hereunder, the subtilisins formed and secreted by Bacillus species in particular should be mentioned as the most important group within the subtilases.

Examples of proteases of the subtilisin type preferably used in washing or cleaning agents are the subtilisins BPN′ and Carlsberg, protease PB92, subtilisins 147 and 309, the alkaline proteases form Bacillus lentus, more particularly from Bacillus lentus DSM 5483, subtilisin DY, and the enzymes thermitase, proteinase K, and proteases TW3 and TW7, which should be classed as subtilases but no longer as subtilisins in the narrower sense. The protease variants having the name BLAP® are derived from the protease from Bacillus lentus DSM 5483. Further usable proteases are, for example, the enzymes available from Novozymes under the tradenames Durazym®, Relase®, Everlase®, Nafizym, Natalase®, Kannase®, and Ovozyme®, the enzymes available from Genencor under the tradenames Purafect®, Purafect® OxP, Purafect® Prime, and Properase®, the enzyme available from Advanced Biochemicals Ltd., Thane, India, under the tradename Protosol®, the enzyme available from Wuxi Snyder Bioproducts Ltd., China, under the tradename Wuxi®, the enzymes available from Amano Pharmaceuticals Ltd., Nagoya, Japan, under the tradenames Proleather® and Protease P®, and the enzyme available from Kao Corp., Tokyo, Japan, under the name Proteinase K-16.

In general, the washing performance of a washing agent decreases with increasing water hardness. Likewise in the case of washing-agent proteases, a loss of performance with increasing water hardness can be observed. The loss of performance can be reduced by incorporating substances that reduce water hardness, such as phosphonates and citrates, which means additional formulation complexity, however.

A performance-increasing effect on washing-agent proteases is described for various polyanionic substances, such as polyacrylates and poly-g-glutamate. However, the corresponding polymer must be incorporated into the formulation in addition to the protease, which causes particular complexity and cost.

Therefore, the problem addressed by the present invention is that of providing a simple-to-formulate system that prevents or at least reduces the loss of performance of washing-agent proteases linked to increasing water hardness.

This problem is solved according to the invention by providing a washing-agent protease modified by the covalent attachment of a polyanion, a protease-polyanion fusion protein.

The modification according to the invention enhances the water hardness tolerance over the starting molecule without a further ingredient having to be changed in addition to the protease.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A protease-polyanion fusion protein, containing a protease comprising an amino acid sequence that is at least 80% identical to one of the sequences indicated in SEQ ID NO. 1 to SEQ ID NO. 5 over the entire length of said one of the amino acid sequences indicated in SEQ ID NO. 1 to SEQ ID NO. 5, and a polyanion.

### BRIEF DESCRIPTION OF THE DRAWINGS

The present invention will hereinafter be described in conjunction with the following drawing FIGURES, wherein like numerals denote like elements, and

FIG. 1 shows the washing performance of the example protease according to the invention with increasing water hardness.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In principle, all proteolytic enzymes that can be used in washing or cleaning agents, particularly subtilases, preferably subtilases whose washing or cleaning performance at least matches that of Savinase (SEQ ID NO. 2) or BPN′ (SEQ ID NO. 3), are proteases that can be modified according to the invention, wherein the cleaning performance is determined in a washing system that contains a washing agent in a dosage between 4.5 and 7.0 grams per liter of washing liquid and the protease, wherein the proteases to be compared are used at the same concentration (with respect to active protein) and the cleaning performance with respect to a blood stain on cotton, more particularly with respect to the blood stain on cotton: product no. 111 available from Eidgenössische Material- and Prüfanstalt (EMPA) Testmaterialien AG, St. Gallen, Switzerland, is determined by measuring the degree of whiteness of the washed textiles, the washing process occurs for 70 minutes at a temperature of 40° C., and the water has a water hardness between 15.5 and 16.5 dGH (degrees of German hardness). The concentration of the protease in the washing agent specified for this washing system is 0.001-0.15 wt %, preferably 0.0015-0.1 wt %, especially preferably 0.01 to 0.075 wt %, with respect to active protein.

A preferred liquid washing agent for such a washing system is composed as follows (all specifications in weight percent): 0.3-0.5% xanthan gum, 0.2-0.4% antifoaming agent, 6-7% glycerol, 0.3-0.5% ethanol, 4-7% FAEOS (fatty alcohol ether sulfate), 24-28% nonionic surfactants, 1% boric acid, 1-2% sodium citrate (dihydrate), 2-4% soda, 14-16% coconut fatty acids, 0.5% HEDP (1-hydroxyethane-(1,1-diphosphonic acid)), 0-0.4% PVP (polyvinylpyrrolidone), 0-0.05% optical brightener, 0-0.001% dye, remainder: demineralized water. The dosage of the liquid washing agent is preferably between 4.5 and 6.0 grams per liter of washing liquid, for example, 4.7, 4.9, or 5.9 grams per liter of washing liquid. The washing is preferably performed in a pH value range of between pH 8 and pH 10.5, preferably between pH 8 and pH 9.

A preferred powdery washing agent for such a washing system is composed as follows (all specifications in weight percent): 10% linear alkylbenzene sulfonate (sodium salt), 1.5% C12-C18 fatty alcohol sulfate (sodium salt), 2.0% C12-C18 fatty alcohol having 7 EO units, 20% sodium carbonate, 6.5% sodium hydrogencarbonate, 4.0% amorphous sodium disilicate, 17% sodium carbonate peroxyhydrate, 4.0% TAED, 3.0% polyacrylate, 1.0% carboxymethyl cellulose, 1.0% phosphonate, 27% sodium sulfate, remainder: foam inhibitors, optical brightener, fragrances. The dosage of the powdery washing agent is preferably between 4.5 and 7.0 grams per liter of washing liquid, for example and especially preferably 4.7 grams per liter of washing liquid, or 5.5, 5.9, or 6.7 grams per liter of washing liquid. The washing is preferably performed in a pH value range of between pH 9 and pH 11.

In the context of the invention, the cleaning performance is determined at 40° C. by using a liquid washing agent as indicated above, wherein the washing process preferably occurs for 70 minutes.

The degree of whiteness, i.e., the brightening of the stains, is determined preferably by means of optical measurement methods, preferably photometrically, as a measure of the cleaning performance. A device suitable for this is, for example, the Minolta CM508d spectrometer. Typically, the devices used for the measurement are calibrated beforehand by means of a white standard, preferably an included white standard.

An especially preferred protease that can be modified according to the invention comprises an amino acid sequence that is at least 80% and increasingly preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, and 99% identical to one of the amino acid sequences indicated in SEQ ID NO. 1 to SEQ ID NO. 5 over the entire length of said one of the amino acid sequences indicated in SEQ ID NO. 1 to SEQ ID NO. 5.

SEQ ID NO. 1 is the sequence of a mature alkaline protease from Bacillus lentus, which has the amino acid glutamic acid (E) at position 99 in the counting according to SEQ ID NO. 1.

The identity of nucleic acid or amino acid sequences is determined by means of a sequence comparison. This sequence comparison is based on the commonly used BLAST algorithm established in the prior art (see, for example, Altschul, S. F., Gish, W., Miller, W., Myers, E. W., & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pp. 3389-3402) and is performed basically by associating similar sequences of nucleotides or amino acids in the nucleic acid or amino acid sequences with each other. A tabular association of the positions in question is referred to as an alignment. A further algorithm available in the prior art is the FASTA algorithm. Sequence comparisons (alignments), particularly multiple sequence comparisons, are created by means of computer programs. For example, the Clustal series (see, for example, Chenna et al. (2003): Multiple sequence alignment with the Clustal series of programs. Nucleic Acid Research 31, 3497-3500), T-Coffee (see, for example, Notredame et al. (2000): T-Coffee: A novel method for multiple sequence alignments. J. Mol. Biol. 302, 205-217), or programs based on these programs or algorithms are frequently used. In the present invention, all sequence comparisons (alignments) were created by means of the computer program Vector NTI® Suite 10.3 (Invitrogen Corporation, 1600 Faraday Avenue, Carlsbad, Calif., USA) with the specified standard parameters, the AlignX module of which for the sequence comparisons is based on ClustalW.

Such a comparison also allows a statement about the similarity of the compared sequences to each other. This similarity is commonly stated in percent identity, i.e., the percentage of identical nucleotides or amino acid residues at the same positions or at positions corresponding to each other in an alignment. The broader concept of homology also takes into consideration conservative amino acid exchanges in the case of amino acid sequences, i.e., amino acids having similar chemical activity, because they usually have similar chemical activity within the protein. Therefore, the similarity of the compared sequences can also be stated as percent homology or percent similarity. Identity and/or homology statements can be made over whole polypeptides or genes or only over individual ranges. Homologs or identical ranges of different nucleic acid or amino sequences are therefore defined by homologies in the sequences. Such ranges often have identical functions. They can be small or comprise only a few nucleotides or amino acids. Such small ranges often have functions that are essential to the overall activity of the protein. Therefore, it can be sensible to relate sequence homologies only to individual, possibly small ranges. However, unless otherwise indicated, identity or homology statements in the present invention relate to the entire length of the indicated nucleic acid or amino acid sequence.

The polyanion (polyanionic polymer) usable according to the invention to modify the protease is preferably a biopolymer constructed from polyamino acids, more particularly a biopolymer constructed completely or predominantly of acidic amino acids (Asp, Glu) and having a negative net charge (formal charge), preferably in the range around pH 7.5. The formal charge is determined by counting the acidic (negatively charged) amino acids of the biopolymer and subtracting the number of basic (positively charged) amino acids therefrom. Thus, a polyanion usable according to the invention can consist exclusively of acidic amino acids, but can also comprise neutral or basic amino acids so long as the net charge remains negative. The usable polyanion is composed of preferably at least 70%, more preferably at least 80%, and especially at least 81%, 82%, 83%, 84%, 85%, 86%, 87%, 88%, 89%, 90%, 91%, 92%, 93%, 94%, 95%, 96%, 97%, 98%, 99%, or 100%, acidic amino acids.

The polyanion can be bonded N- or C-terminally to the protease or can be inserted into the protease molecule in place of a loop of the protease molecule, wherein the C-terminal bonding is very especially preferred. Covalent attachment by means of a peptide bond is preferred. The polyanion preferably has a length of 10 to 1000, more particularly 10 to 500, preferably 10 to 100, especially preferably 20 to 50, very especially preferably 25 to 40, and most preferably approximately 30, amino acid residues, more particularly aspartate and/or glutamate residues.

According to the invention, the polyanion can be coexpressed together with the protease or attached to the protease after the protease has been expressed. The coexpression of the protease and the polyanion, as described later, is preferred, because subsequent attachment requires an additional step.

Therefore, a protease-polyanion fusion protein according to the present invention is a protease modified by the covalent attachment of a polyanion.

The invention also relates to a nucleic acid that codes for a modified protease according to the invention, and to a vector containing such a nucleic acid, more particularly a cloning vector or an expression vector.

These can be DNA or RNA molecules. They can be present as a single strand, as a single strand complementary to said single strand, or as a double strand. Particularly in the case of DNA molecules, the sequences of both complementary strands in all three possible reading frames must be taken into consideration. Furthermore, it must be considered that different codons, i.e., base triplets, can code for the same amino acids, and therefore several different nucleic acids can code for a certain amino acid sequence. Because of this degeneracy of the genetic code, all nucleic acid sequences that can code for one of the proteases described above are included in this subject matter of the invention. A person skilled in the art is capable of determining these nucleic acid sequences beyond all doubt, because defined amino acids can be associated with individual codons despite the degeneracy of the genetic code. Therefore, on the basis of an amino acid sequence, a person skilled in the art can determine nucleic acids that code for this amino acid sequence without trouble. Furthermore, one or more codons can be replaced by synonymous codons in the nucleic acids according to the invention. This aspect relates in particular to the heterologous expression of the enzymes according to the invention. Every organism, for example a host cell of a production strain, has a certain codon use. By “codon use,” the translation of the genetic code into amino acids by the particular organism is understood. Bottlenecks can occur in protein biosynthesis if the codons lying on the nucleic acid are accompanied by a comparatively low number of charged tRNA molecules in the organism. This has the result that a codon is translated less efficiently in the organism than a synonymous codon that codes for the same amino acid, although the codons code for the same amino acid. Because of the presence of a higher number of tRNA molecules for the synonymous codon, the synonymous codon can be translated more efficiently in the organism.

It is possible for a person skilled in the art to produce, on the basis of known DNA sequences and/or amino acid sequences, the corresponding nucleic acids to the point of complete genes by means of methods that are now well known, such as chemical synthesis or polymerase chain reaction (PCT) in conjunction with standard methods of molecular biology and/or protein chemistry. Such methods are known, for example, from Sambrook, J., Fritsch, E. F., and Maniatis, T. 2001. Molecular cloning: a laboratory manual, 3rd Edition Cold Spring Laboratory Press.

In the sense of the present invention, the term “vectors” means elements that consist of nucleic acids and that contain a nucleic acid according to the invention as a characterizing nucleic acid region. Vectors are able to establish said nucleic acid according to the invention as a stable genetic element in a species or a cell line over several generations or cells divisions. Particularly in the case of use in bacteria, vectors are specific plasmids, i.e., circular genetic elements. In the context of the present invention, a nucleic acid according to the invention is cloned into a vector. The vectors include, for example, vectors having bacterial plasmids, viruses, or bacteriophages as their origin, or predominantly synthetic vectors or plasmids having elements of widely different origin. By means of the further present genetic elements, vectors are able to establish themselves as stable units in the host cells in question over several generations. They can be present in extrachromosomal form as separate units or integrated into a chromosome or chromosomal DNA.

Expression vectors comprise nucleic acid sequences that enable them to replicate in the host cells containing them, preferably microorganisms, especially preferably bacteria, and to cause a contained nucleic acid to be expressed there. The expression is influenced in particular by the one or more promoters that regulate the transcription. In principle, the expression can occur by means of the natural promoter originally located before the nucleic acid to be expressed but can also occur by means of a promoter of the host cell provided on the expression vector or by means of a modified promoter or completely different promoter of a different organism or of a different host cell. In the present case, at least one promoter for the expression of a nucleic acid according to the invention is provided and is used for the expression of said nucleic acid. Furthermore, expression vectors can be controllable, for example by changing the cultivation conditions or when a certain cell density of the host cells containing the expression vectors is reached or by adding certain substances, particularly activators of the gene expression. An example of such a substance is the galactose derivative isopropyl-β-D-thiogalactopyranoside (IPTG), which is used as an activator of the bacterial lactose operon (lac operon). In contrast to expression vectors, the contained nucleic acid in cloning vectors is not expressed.

The invention also relates to a non-human host cell that contains a nucleic acid according to the invention or a vector according to the invention or contains a modified protease according to the invention, more particularly a non-human host cell that secretes the protease into the medium surrounding the host cell. A nucleic acid according to the invention or a vector according to the invention is preferably introduced into a microorganism in a transformation process, which microorganism then constitutes a host cell according to the invention. Alternatively, individual components, i.e., nucleic acid parts or nucleic acid fragments of a nucleic acid according to the invention can also be introduced into a host cell in such a way that the resulting host cell contains a nucleic acid according to the invention or a vector according to the invention. This procedure is suitable especially if the host cell already contains one or more components of a nucleic acid according to the invention or of a vector according to the invention and the further components are then added accordingly. Methods for transforming cells are established in the prior art and are well known to a person skilled art. In principle, all cells, i.e., prokaryotic and eukaryotic cells, are suitable as host cells. Host cells that can be advantageously handled genetically, for example with regard to the transformation by means of the nucleic acid or the vector and the stable establishment thereof, are preferred, for example single-cell fungi or bacteria. Furthermore, preferred host cells are distinguished by good microbiological and biotechnological manageability. This relates to, for example, ease of cultivation, high growth rates, low requirements for fermentation media, and good production rates and secretion rates for foreign proteins. Preferred host cells according to the invention secrete the (transgenically) expressed protein into the medium surrounding the host cells. Furthermore, the proteases can be modified by the cells that produce them after the production of the proteases, for example by the attachment of sugar molecules, formylations, and aminations. Such post-translational modifications can functionally influence the protease.

Additional preferred embodiments are host cells whose activity can be controlled on the basis of genetic regulation elements, which are provided on the vector for example but can also be present in these cells from the outset. Expression of these can be induced, for example, by the controlled addition of chemical compounds that act as activators, by changing the cultivation conditions, or when a certain cell density is reached. This enables economical production of the proteins according to the invention. An example of such a compound is IPTG, as described above.

Preferred host cells are prokaryotic or bacterial cells. Bacteria are distinguished by short generation times and low requirements for the cultivation conditions. Thus, economical cultivation methods or production methods can be established. In addition, a person skilled in the art has a wealth of experience at his disposal with regard to bacteria in fermentation technology. For a specific production, gram-negative or gram-positive bacteria can be suitable for a wide range of reasons to be determined experimentally in each individual case, such as nutrient sources, product formation rate, and time requirement.

In the case of gram-negative bacteria such as Escherichia coli, a large number of proteins is secreted into the periplasmic space, i.e., into the compartment between the two membranes enclosing the cells. This can be advantageous for specific applications. Furthermore, gram-negative bacteria also can be designed in such a way that they discharge the expressed proteins not only into the periplasmic space but also into the medium surrounding the bacterium. In contrast, gram-positive bacteria, such as Bacilli or actinomycetes or other representatives of the Actinomycetales do not have an outer membrane, and therefore secreted proteins are immediately discharged into the medium surrounding the bacteria, generally the nutrient medium, from which the expressed proteins can be purified. They can be directly isolated from the medium or processed further. In addition, gram-positive bacteria are related or identical to most organisms of origin for technically important enzymes and usually form comparable enzymes themselves, and therefore they have similar codon use and their protein synthesis apparatus is of course oriented accordingly.

Host cells according to the invention can be changed with regard to their requirements for the culture conditions, can have other or additional selectable markers, or can express other or additional proteins. In particular, said host cells can also be host cells that transgenically express several proteins or enzymes.

In principle, the present invention can be applied to all microorganisms, particularly to all microogranisms capable of fermentation, especially preferably to those of the genus Bacillus, and has the result that proteins according to the invention can be produced by using such microorganisms. Such microorganisms are then host cells in the sense of the invention.

In a further embodiment of the invention, the host cell is characterized in that it is a bacterium, preferably a bacterium selected from the group of genera comprising Escherichia, Klebsiella, Bacillus, Staphylococcus, Corynebacterium, Arthrobacter, Streptomyces, Stenotrophomonas, and Pseudomonas, more preferably a bacterium selected from the group comprising Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxydans, Streptomyces lividans, Streptomyces coelicolor and Stenotrophomonas maltophilia.

However, the host cell can also be a eukaryotic cell, which is characterized in that it has a nucleus. Therefore, the invention also relates to a host cell that is characterized in that it has a nucleus. In contrast to prokaryotic cells, eukaryotic cells are capable of post-translationally modifying the formed protein. Examples are fungi such as actinomycetes or yeasts such as Saccharomyces or Kluyveromyces. This can be especially advantageous when, for example, the proteins should experience, in conjunction with their synthesis, specific modifications that such systems enable. The modifications that eukaryotic systems perform especially in conjunction with the protein synthesis include, for example, the binding of low-molecular-weight compounds such as membrane anchors or oligosaccharides. Such oligosaccharide modifications can be desirable, for example, in order to reduce the allergenicity of an expressed protein. Coexpression with the enzymes naturally formed by such cells, such as cellulases or lipases, can also be advantageous. Furthermore, thermophilic-fungus expressions systems, for example, are especially suitable for the expression of temperature-resistant proteins or variants.

The host cells according to the invention are cultivated and fermented in a typical manner, for example in discontinuous or continuous systems. In the first case, a suitable nutrient medium is inoculated with the host cells and the product is harvested from the medium after a time period to be determined experimentally. Continuous fermentations are characterized by the attainment of a steady state in which, over a comparatively long time period, some cells die off but new cells are also grown and at the same time the formed protein can be withdrawn from the medium.

Host cells according to the invention are preferably used to produce modified proteases according to the invention. Therefore, the invention also relates to a method for producing a protease, comprising

a) cultivating a host cell according to the invention,

b) isolating the protease from the culture medium or from the host cell.

This subject matter of the invention preferably comprises fermentation methods. Fermentation methods are known per se from the prior art and are the actual large-scale production step, generally followed by a suitable method for purifying the produced product, for example the protease according to the invention. All fermentation methods based on a corresponding method for producing a protease according to the invention are embodiments of this subject matter of the invention.

In particular, fermentation methods that are characterized in that the fermentation is performed by means of a supply strategy are considered. In this case, the media constituents that are consumed by the continuous cultivation are added. Considerable increases both in the cell density and in the cell mass or dry mass and/or in particular in the activity of the protease of interest can thereby be achieved. Furthermore, the fermentation can also be designed in such a way that undesirable metabolic products are filtered out or neutralized by addition of buffer or suitable counterions.

The produced protease can be harvested from the fermentation medium. Such a fermentation method is preferred over isolation of the protease from the host cell, i.e., over product processing from the cell mass (dry mass), but requires that suitable host cells or one or more suitable secretion markers or secretion mechanisms and/or transport systems are provided so that the host cells secrete the protease into the fermentation medium. Without secretion, the protease can alternatively be isolated from the host cell, i.e., can be purified from the cell mass, for example by precipitation with ammonium sulfate or ethanol, or by chromatographic purification.

All the facts presented above can be combined into methods in order produce modified proteases according to the invention.

The invention also relates to an agent that is characterized in that the agent contains a modified protease according to the invention as described above. The agent is preferably a washing or cleaning agent. Because modified proteases according to the invention have advantageous cleaning performance, particularly on stains containing blood, the agents are particularly suitable and advantageous for removing such stains.

This subject matter of the invention includes all conceivable types of washing or cleaning agent, both concentrates and agents to be used undiluted, for use on a commercial scale, in a washing machine, or in washing or cleaning by hand. Included are, for example, washing agents for textiles, carpets, or natural fibers, for which the designation “washing agent” is used. Also included are, for example, dishwashing agents for dishwashers or manual dishwashing agents or cleaners for hard surfaces such as metal, glass, porcelain, ceramic, tiles, stone, painted surfaces, plastics, wood, or leather, for which the designation “cleaning agent” is used, i.e., in addition to manual and machine dishwashing agents, also scouring agents, glass cleaners, and fragrant toilet rim blocks, for example. The washing and cleaning agents in the context of the invention also include washing aids, which are added to the actual washing agent in the washing of textiles manually or by machine in order to achieve further action. Furthermore, the washing and cleaning agents in the context of the invention also include textile pretreatment and posttreatment agents, i.e., agents with which the laundry item is brought into contact before the actual washing, for example in order to loosen tenacious soiling, and agents that give the items to be washed further desirable properties such as pleasant texture, freedom from wrinkles, or low static charge, in a step following the actual textile washing.

The active-protein-related percentage of the protease according to the invention by weight with respect to the total weight of washing or cleaning agents according to the invention is preferably 0.005 to 1.0 wt %, preferably 0.01 to 0.5 wt %, and particularly 0.02 to 0.2 wt %. The protein concentration can be determined by means of known methods, for example the BCA method (bicinchoninic acid; 2,2′-biquinolinyl-4,4′-dicarboxylic acid) or the Biuret method (A. G. Gornall, C. S. Bardawill, and M. M. David, J. Biol. Chem., 177 (1948), pp. 751-766). In this regard, the active protein concentration is determined titrating the active centers by using a suitable irreversible inhibitor (for example, phenylmethylsulfonyl fluoride (PMSF) for proteases) and determining the residual activity (see M. Bender et al., J. Am. Chem. Soc. 88, 24 (1966), pp. 5890-5913).

The washing or cleaning agents according to the invention can contain further enzymes. For example, lipases or cutinases, particularly because of the triglyceride-cleaving activities thereof, but also in order to produce peracids in situ from suitable precursors, can be used as further enzymes. These include, for example, the lipases originally available from Humicola lanuginosa (Thermomyces lanuginosus) or further developed, particularly such having the amino acid exchange D96L. These include, for example, the lipases originally available from Humicola lanuginosa (Thermomyces lanuginosus) or further developed, particularly such having one or more amino acid exchanges in positions D96L, T213R, and/or N233R, especially preferably T213R and N233R, starting from the stated lipase. Furthermore, the cutinases originally isolated from Fusarium solani pisi and Humicola insolens can be used, for example. Also usable are lipases or cutinases whose starting enzymes were originally isolated from Pseudomonas mendocina and Fusarium solanii.

The agents according to the invention can also contain cellulases or hemicellulases such as mannanases, xanthan lyases, pectin lyases (=pectinases), pectinesterases, pectate lyases, xyloglucanases (=xylanases), pullulanases, or β-glucanases.

According to the invention, oxidoreductases, for example oxidases, oxygenases, catalases, peroxidases, such as haloperoxidases, chloroperoxidases, bromoperoxidases, lignin peroxidases, glucose peroxidases, or manganese peroxidases, dioxygenases, or laccases (phenol oxidases, polyphenol oxidases), can be used to increase the bleaching action. Preferably organic, especially preferably aromatic, compounds that interact with the enzymes are advantageously additionally added in order to intensify the activity of the oxidoreductases in question (enhancers) or in order to ensure the electron flow in the event of greatly differing redox potentials between the oxidizing enzymes and the stains (mediators).

Amylases can also be used as further enzymes. Synonymous terms can be used for amylases, for example 1,4-alpha-D-glucan glucanohydrolase or glycogenase. Amylases preferred according to the invention are α-amylases. Decisive for whether an enzyme is an α-amylase in the sense of the invention is the ability of the enzyme to hydrolyze α (1-4) glycosidic bonds in the amylose of the starch.

Examples of amylases are the α-amylases from Bacillus licheniformis, from Bacillus amyloliquefaciens, or from Bacillus stearothermophilus, and in particular also the further developments thereof enhanced for use in washing or cleaning agents. The enzyme from Bacillus licheniformis is available from Novozymes under the name Termamyl® and from Danisco/Genencor under the name Purastar® ST. Products of the further development of these α-amylases are available from Novozymes under the tradenames Duramyl® and Termamyl® ultra, from Danisco/Genencor under the name Purastar® OxAm, and from Daiwa Seiko Inc., Tokyo, Japan, as Keistase®. The α-amylase from Bacillus amyloliquefaciens is sold by Novozymes under the name BAN®, and derived variants of the α-amylase from Bacillus stearothermophilus are sold likewise by Novozymes under the names BSG® and Novamyl®. Furthermore, the α-amylase from Bacillus sp. A 7-7 (DSM 12368) and the cyclodextrin glucanotransferase (CGTase) from Bacillus agaradherens (DSM 9948) should emphasized for this purpose. Likewise, fusion products of all mentioned molecules can be used. Furthermore, the further developments of the α-amylase from Aspergillus niger and A. oryzae available from Novozymes under the tradename Fungamyl® are suitable. Further advantageously usable commercial products are, for example, Amylase-LT® and Stainzyme® or Stainzyme Ultra® or Stainzyme Plus®; the latter are likewise from Novozymes. Variants of these enzymes that can be obtained by point mutations also can be used according to the invention. Especially preferred amylases are disclosed in the laid-open international applications WO 00/60060, WO 03/002711, WO 03/054177, and WO 07/079938, the disclosure of which is therefore expressly referred to or the disclosure content of which is expressly incorporated into the present invention.

The active-protein-related percentage of the further enzymes by weight with respect to the total weight of preferred washing or cleaning agents is preferably 0.0005 to 1.0 wt %, preferably 0.001 to 0.5 wt %, and particularly 0.002 to 0.2 wt %.

The washing or cleaning agents can contain cleaning-active substances in addition to the ingredients previously described, wherein substances from the group comprising surfactants, builders, polymers, glass corrosion inhibitors, corrosion inhibitors, fragrances, and perfume carriers are preferred. These preferred ingredients are described in more detail below.

A preferred constituent of the washing or cleaning agents according to the invention are the nonionic surfactants, wherein nonionic surfactants of the general formula R¹—CH(OH)CH₂O-(AO)_{w}-(A′O)ₓ-(A″O)_{y}-(A′″O)_{z}—R², in which
R¹ represents a straight-chain or branched, saturated or mono- or polyunsaturated C₆₋₂₄ alkyl or alkenyl residue;
R² represents a linear or branched hydrocarbon residue having 2 to 26 carbon atoms;
A, A′, A″, and A′″ represent, independently of each other, a residue from the group
—CH₂CH₂—, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃),
w, x, y, and z represent values between 0.5 and 120, wherein x, y, and/or z can also be 0,
   are preferred.

By adding the aforementioned nonionic surfactants of the general formula R¹—CH(OH)CH₂O-(AO)_{w}-(A′O)ₓ-(A″O)_{y}-(A′″O)_{z}—R², also referred to as “hydroxy mixed ethers” below, the cleaning performance of enzyme-containing preparations according to the invention can be significantly enhanced, namely both in comparison to surfactant-free systems and in comparison to systems that contain alternative nonionic surfactants, for example from the group of the polyalkoxylated fatty alcohols.

By using these nonionic surfactants with one or more free hydroxyl groups on one or both terminal alkyl residues, the stability of the enzymes contained in the washing or cleaning agent preparations according to the invention can be significantly enhanced.

In particular, end-capped poly(oxyalkylated) nonionic surfactants are preferred which, in accordance with the formula R¹O[CH₂CH₂O]ₓCH₂CH(OH)R², comprise, in addition to a residue R¹, which represents linear or branched, saturated or unsaturated, aliphatic or aromatic hydrocarbon residues having 2 to 30 carbon atoms, preferably having 4 to 22 carbon atoms, also a linear or branched, saturated or unsaturated, aliphatic or aromatic hydrocarbon residue R² having 1 to 30 carbon atoms, wherein x represents values between 1 and 90, preferably values between 30 and 80, and particularly values between 30 and 60.

Especially preferred are surfactants of the formula R¹O[CH₂CH(CH₃)O]ₓ[CH₂CH₂O]_{y}CH₂CH(OH)R², in which R¹ represents a linear or branched aliphatic hydrocarbon residue having 4 to 18 carbon atoms or mixtures thereof, R² represents a linear or branched hydrocarbon residue having 2 to 26 carbon atoms or mixtures thereof, x represents values between 0.5 and 1.5, and y represents a value of at least 15. The group of these nonionic surfactants includes, for example, the C₂₋₂₆ fatty alcohol (PO)₁-(EO)₁₅₋₄₀-2-hydroxyalkylethers, in particular also the C₈₋₁₀ fatty alcohol (PO)₁-(EO)₂₂-2-hydroxydecylethers.

Furthermore, end-capped poly(oxyalkylated) nonionic surfactants of the formula R¹O[CH₂CH₂O]ₓ[CH₂CH(R³)O]_{y}CH₂CH(OH)R², in which R¹ and R² represent, independently of each other, a linear or branched, saturated or mono- or polyunsaturated hydrocarbon residue having 2 to 26 carbon atoms, R³ is selected, independently in each case, from —CH₃, —CH₂CH₃, —CH₂CH₂—CH₃, —CH(CH₃)₂, but preferably represents —CH₃, and x and y represent, independently of each other, values between 1 and 32, are especially preferred, wherein nonionic surfactants with R³=—CH₃ and values of 15 to 32 for x and of 0.5 and 1.5 for y are very especially preferred.

Further nonionic surfactants that can be used with preference are the end-capped poly(oxyalkylated) nonionic surfactants of the formula R¹O[CH₂CH(R³)O]ₓ[CH₂]ₖCH(OH)[CH₂]ⱼOR², in which R¹ and R² represent linear or branched, saturated or unsaturated, aliphatic or aromatic hydrocarbon residues having 1 to 30 carbon atoms, R³ represents H or a methyl, ethyl, n-propyl, isopropyl, n-butyl, 2-butyl, or 2-methyl-2-butyl residue, x represents values between 1 and 30, and k and j represent values between 1 and 12, preferably between 1 and 5. If the value of x is ≧2, each R³ in the formula R¹O[CH₂CH(R³)O]ₓ[CH₂]ₖCH(OH)[CH₂]ⱼOR² above can be different. R¹ and R² are preferably linear or branched, saturated or unsaturated, aliphatic or aromatic hydrocarbon residues having 6 to 22 carbon atoms, wherein residues having 8 to 18 C atoms are especially preferred. For the residue R³, H, —CH₃, or —CH₂CH₃ is especially preferred. Especially preferred values for x lie in the range of 1 to 20, particularly 6 to 15.

As described above, each R³ in the formula above can be different if x is ≧2. The alkylene oxide unit in the square brackets can thereby be varied. If x represents 3, for example, the residue R³ can be selected in order to form ethylene oxide units (R³═H) or propylene oxide units (R³═CH₃), which can be joined to each other in any sequence, for example (EO)(PO)(EO), (EO)(EO)(PO), (EO)(EO)(EO), (PO)(EO)(PO), (PO)(PO)(EO), and (PO)(PO)(PO). The value 3 for x has been selected as an example and definitely can be greater, wherein the range of variation increases with increasing x values and includes, for example, a large number of (EO) groups combined with a low number of (PO) groups or vice versa.

Especially preferred end-capped poly(oxyalkylated) alcohols of the formula above have values of k=1 and j=1, the formula above thus being simplified to R¹O[CH₂CH(R³)O]ₓCH₂CH(OH)CH₂OR². In the last-mentioned formula, R¹, R², and R³ are defined as above and x represents numbers from 1 to 30, preferably from 1 to 20, and particularly from 6 to 18. Especially preferred are surfactants in the case of which the residues R¹ and R² have 9 to 14 C atoms, R³ stands for H, and x assumes values of 6 to 15.

Finally, the nonionic surfactants of the general formula R¹—CH(OH)CH₂O-(AO)_{w}—R², in which
R¹ represents a straight-chain or branched, saturated or mono- or polyunsaturated C₆₋₂₄ alkyl or alkenyl residue;
R² represents a linear or branched hydrocarbon residue having 2 to 26 carbon atoms;
A represents a residue from the group CH₂CH₂, —CH₂CH₂—CH₂, —CH₂—CH(CH₃); and
w represents values between 1 and 120, preferably 10 to 80, particularly 20 to 40, have proven to be especially effective.
   The group of these nonionic surfactants includes, for example, the C₄₋₂₂ fatty alcohol (EO)₁₀₋₈₀-2-hydroxyalkylethers, in particular also the C₈₋₁₀ fatty alcohol (EO)₂₂-2-hydroxydecylethers and the C₄₋₂₂ fatty alcohol (EO)₄₀₋₈₀-2-hydroxyalkylethers.

Preferred washing or cleaning agents are characterized in that the washing or cleaning agent contains at least one nonionic surfactant, preferably one nonionic surfactant from the group of the hydroxy mixed ethers, wherein the percentage of the nonionic surfactant by weight with respect to the total weight of the washing or cleaning agent is preferably 0.2 to 10 wt %, preferably 0.4 to 7.0 wt %, and particularly 0.6 to 6.0 wt %.

Preferred washing or cleaning agents according to the invention for use in machine dishwashing methods contain further surfactants, particularly amphoteric surfactants, in addition to the previously described nonionic surfactants. However, the percentage of anionic surfactants with respect to the total weight of these washing or cleaning agents is preferably limited. Thus, preferred machine dishwashing agents are characterized in that they contain, with respect to the total weight thereof, less than 5.0 wt %, preferably less than 3.0 wt %, especially preferably less than 2.0 wt %, of anionic surfactant. The use of anionic surfactants in a larger amount is forgone particularly in order to avoid excessive foaming.

An additional preferred constituent of washing or cleaning agents according to the invention are complexing agents. Especially preferred complexing agents are the phosphonates. The complexing phosphonates comprise a series of different compounds such as diethylenetriamine penta(methylene phosphonic acid) (DTPMP) in addition to the 1-hydroxyethane-1,1-diphosphonic acid. In particular, hydroxyalkanephosphonates and aminoalkanephosphonates are preferred in this application. Among the hydroxyalkanephosphonates, 1-hydroxyethane-1,1-diphosphonate (HEDP) is especially important as a co-builder. It is preferably used as a sodium salt, wherein the disodium salt reacts in a neutral manner and the tetrasodium salt reacts in an alkaline manner (pH 9). Preferably ethylenediamine tetramethylene phosphonate (EDTMP), diethylenetriamine pentamethylene phosphonate (DTPMP), and their higher homologs are considered as aminoalkanephosphonates. They are preferably used in the form of the sodium salts that react in a neutral manner, e.g., as hexasodium salt of EDTMP or as hepta- and octasodium salt of DTPMP. From the class of the phosphonates, preferably HEDP is used as a builder. The aminoalkanephosphonates additionally have a pronounced heavy metal binding capability. Accordingly, it can be preferred, particularly if the agents also contain bleaches, to use aminoalkanephosphonates, particularly DTPMP, or mixtures of the mentioned phosphonates.

A washing or cleaning agent preferred in the context of this application contains one or more phosphonates from the group comprising
a) aminotris(methylenephosphonic acid) (ATMP) and/or salts thereof;
b) ethylenediamine tetra(methylene phosphonic acid) (EDTMP) and/or salts thereof;
c) diethylenetriamine penta(methylene phosphonic acid) (DTPMP) and/or salts thereof;
d) 1-hydroxyethane-1,1-diphosphonic acid (HEDP) and/or salts thereof;
e) 2-phosphonobutane-1,2,4-tricarboxylic acid (PBTC) and/or salts thereof;
f) hexamethylenediamine tetra(methylene phosphonic acid) (HDTMP) and/or salts thereof;
g) nitrilotris(methylene phosphonic acid) (NTMP) and/or salts thereof.

Especially preferred are washing or cleaning agents that contain 1-hydroxyethane-1,1-diphosphonic acid (HEDP) or diethylenetriamine penta(methylene phosphonic acid) (DTPMP) as a phosphonate. Of course, the washing or cleaning agents according to the invention can contain two or more different phosphonates. Preferred washing or cleaning agents according to the invention are characterized in that the washing or cleaning agent contains at least one complexing agent from the group of the phosphonates, preferably 1-hydroxyethane-1,1-diphosphonate, wherein the percentage of the phosphonate by weight with respect to the total weight of the washing or cleaning agent is preferably between 0.1 and 8.0 wt %, preferably between 0.2 and 5.0 wt %, and particularly between 0.5 and 3.0 wt %.

The washing or cleaning agents according to the invention preferably also contain a builder. Builders include, in particular, the silicates, carbonates, organic co-builders, and, where there are no ecological prejudgments against the use thereof, also the phosphates.

Among the large number of commercially available phosphates, the alkali metal phosphates are of greatest importance for the agents according to the invention, with special preference for pentasodium triphosphate, Na₅P₃O₁₀ (sodium tripolyphosphate), or pentapotassium triphosphate, K₅P₃O₁₀ (potassium tripolyphosphate). If phosphates are used as cleaning-active substances in the washing or cleaning agent in the context of the present invention, preferred agents contain this/these phosphate(s), preferably pentapotassium triphosphate, wherein the percentage of the phosphate by weight with respect to the total weight of the washing or cleaning agent is preferably between 5.0 and 40 wt %, preferably between 10 and 30 wt %, and particularly between 12 and 25 wt %.

In particular, polycarboxylates/polycarboxylic acids, polymeric polycarboxylates, aspartic acid, polyacetals, dextrins, further organic co-builders, and phophonates should be mentioned as organic co-builders. These material classes are described below.

Usable organic builder substances are, for example, the polycarboxylic acids, which can be used in the form of the free acid and/or in the form of the sodium salts thereof, wherein polycarboxylic acids are understood to include carboxylic acids that bear more than one acid function. These are, for example, citric acid, adipic acid, succinic acid, glutaric acid, malic acid, tartaric acid, maleic acid, fumaric acid, sugar acids, amino carboxylic acids, nitrilotriacetic acid (NTA), provided that such a use is not subject to complaints for ecological reasons, and mixtures thereof. In addition to their builder effect, the free acids typically also have the characteristic of an acidification component and therefore also serve to set a lower and milder pH value of washing or cleaning agents. In particular, citric acid, succinic acid, glutaric acid, adipic acid, gluconic acid, and any mixtures thereof should be mentioned in this regard. Citric acid or salts of citric acid are especially preferably used as a builder substance. Additional especially preferred builder substances are selected from methyl glycine diacetic acid (MGDA), glutamic acid diacetate (GLDA), aspartic acid diacetate (ASDA), hydroxyethyl iminodiacetate (HEIDA), iminodisuccinate (IDS), and ethylenediamine disuccinate (EDDS), carboxymethyl inulin, and polyaspartate.

Further polymeric polycarboxylates are suitable as builders. These are, for example, the alkali metal salts of polyacrylic acid or of polymethacrylic acid, such as those having a relative molecular mass of 500 to 70000 g/mol.

Within the meaning of this document, the molar masses stated for polymeric polycarboxylates are weight-average molar masses M_{w} of the acid form, which were in general determined by means of gel permeation chromatography (GPC), wherein a UV detector was used. The measurement was performed against an external polyacrylic acid standard that provides realistic molecular weight values because of its structural relationship to the examined polymers. These data deviate significantly from the molecular weight data for which polystyrene sulfonic acids were used as a standard. The molar masses measured against polystyrene sulfonic acids are generally significantly higher than the molar masses stated in this document.

Suitable polymers are, in particular, polyacrylates, which preferably have a molecular mass of 2000 to 20000 g/mol. In turn, because of their superior solubility, the short-chain polyacrylates having molar masses of 2000 to 10000 g/mol, and especially preferably 3000 to 5000 g/mol, can be preferred among this group.

Also suitable are copolymeric polycarboxylates, particularly those of acrylic acid with methacrylic acid and of acrylic acid or methacrylic acid with maleic acid. Copolymers of acrylic acid with maleic acid containing 50 to 90 wt % of acrylic acid and 50 to 10 wt % of maleic acid have proven to be especially suitable. Their relative molecular mass, with respect to free acids, is generally 2000 to 70000 g/mol, preferably 20000 to 50000 g/mol, and particularly 30000 to 40000 g/mol.

Oxydisuccinates and other derivatives of disuccinates, preferably ethylenediamine di succinate, are additional suitable co-builders. Ethylenediamine-N,N′-disuccinate (EDDS) is preferably used in the form of the sodium or magnesium salts thereof. Also preferred in this context are glycerol disuccinates and glycerol trisuccinates.

To improve the cleaning performance and/or to set the viscosity, preferred washing or cleaning agents contain at least one hydrophobically modified polymer, preferably a hydrophobically modified carboxylic-acid-group-containing polymer, wherein the percentage of the hydrophobically modified polymer by weight with respect to the total weight of the washing or cleaning agent is preferably 0.1 to 10 wt %, preferably between 0.2 and 8.0 wt %, and particularly between 0.4 to 6.0 wt %.

In addition to the previously described builders, cleaning-active polymers can be contained in the washing or cleaning agent. The percentage of the cleaning-active polymers by weight with respect to the total weight of machine washing or cleaning agents according to the invention is preferably 0.1 to 20 wt %, preferably 1.0 to 15 wt %, and particularly 2.0 to 12 wt %.

Preferably sulfonic-acid-group-containing polymers, particularly from the group of the copolymeric polysulfonates, are used as cleaning-active polymers. These copolymeric polysulfonates contain, in addition to sulfonic-acid-group-containing monomer(s), at least one monomer from the group of the unsaturated carboxylic acids.

Unsaturated carboxylic acids of the formula R¹(R²)C═C(R³)COOH, in which R¹ to R³ stand, independently of each other, —H, —CH₃, for a straight-chain or branched saturated alkyl residue having 2 to 12 carbon atoms, for a straight-chain or branched, mono- or polyunsaturated alkenyl residue having 2 to 12 carbon atoms, for alkyl or alkenyl residues as defined above substituted with —NH₂, —OH, or —COOH, or for —COOH or —COOR⁴, wherein R⁴ is a saturated or unsaturated, straight-chain or branched hydrocarbon residue having 1 to 12 carbon atoms, are especially preferably used as unsaturated carboxylic acid(s).

Especially preferred unsaturated carboxylic acids are acrylic acid, methacrylic acid, ethacrylic acid, α-chloroacrylic acid, α-cyanoacrylic acid, crotonic acid, α-phenylacrylic acid, maleic acid, maleic anhydride, fumaric acid, itaconic acid, citraconic acid, methylenemalonic acid, sorbic acid, cinnamic acid, or mixtures thereof. Of course, the unsaturated dicarboxylic acids can also be used.

Among the sulfonic-acid-group-containing monomers, those of the formula R⁵(R⁶)C═C(R⁷)—X—SO₃H are preferred, in which R⁵ to R⁷ stand, independently of each other, for —H, —CH₃, for a straight-chain or branched saturated alkyl residue having 2 to 12 carbon atoms, for a straight-chain or branched, mono- or polyunsaturated alkenyl residue having 2 to 12 carbon atoms, for alkyl or alkenyl residues substituted with —NH₂, —OH, or —COOH, or for —COOH or —COOR⁴, wherein R⁴ is a saturated or unsaturated, straight-chain or branched hydrocarbon residue having 1 to 12 carbon atoms, and X represents an optionally present spacer group, which is selected from —(CH₂)ₙ— with n=0 to 4, —COO—(CH₂)ₖ— with k=1 to 6, —C(O)—NH—C(CH₃)₂—, —C(O)—NH—C(CH₃)₂—CH₂—, and —C(O)—NH—CH(CH₂CH₃)—.

Among these monomers, those of the formulas H₂C═CH—X—SO₃H, H₂C═C(CH₃)—X—SO₃H and HO₃S—X—(R⁶)C═C(R⁷)—X—SO₃H are preferred, in which R⁶ and R⁷ are selected independently of each other from —H, —CH₃, —CH₂CH₃, —CH₂CH₂CH₃, —CH(CH₃)₂ and X represents an optionally present spacer group, which is selected from —(CH₂)ₙ— with n=0 to 4, —COO—(CH₂)ₖ— with k=1 to 6, —C(O)—NH—C(CH₃)₂—, —C(O)—NH—C(CH₃)₂—CH₂—, and —C(O)—NH—CH(CH₂CH₃)—.

Especially preferred sulfonic-acid-group-containing monomers are 1-acrylamido-1-propanesulfonic acid, 2-acrylamido-2-propanesulfonic acid, 2-acrylamido-2-methyl-1-propane sulfonic acid, 2-methacrylamido-2-methyl-1-propane sulfonic acid, 3-methacrylamido-2-hydroxy-propane sulfonic acid, allyl sulfonic acid, methallyl sulfonic acid, allyloxy benzene sulfonic acid, methallyloxy benzene sulfonic acid, 2-hydroxy-3-(2-propenyloxy)propane sulfonic acid, 2-methyl-2-propene-1-sulfonic acid, styrene sulfonic acid, vinylsulfonic acid, 3-sulfopropyl acrylate, 3-sulfopropyl methacrylate, sulfomethacrylamide, sulfomethylmethacrylamide, mixtures of the mentioned acids, or water-soluble salts of the mentioned acids.

The sulfonic acid groups can be present completely or partially in neutralized form in the polymers. The use of partial or fully neutralized sulfonic-acid-group-containing copolymers is preferred according to the invention.

The molar mass of the sulfo-copolymers preferably used according to the invention can be varied to adapt the properties of the polymers to the desired use. Preferred machine dishwashing agents are characterized in that the copolymers have molar masses of 2000 to 200,000 gmol⁻¹, preferably 4000 to 25,000 gmol⁻¹, and particularly 5000 to 15,000 gmol⁻¹.

In an additional preferred embodiment, the copolymers comprise, in addition to a carboxyl-group-containing monomer and a sulfonic-acid-group-containing monomer, at least one nonionic, preferably hydrophobic monomer. By using these hydrophobically modified polymers, it was possible, in particular, to improve the rinsing performance of machine dishwashing agents according to the invention.

Washing or cleaning agents containing a copolymer comprising

i) carboxylic-acid-group-containing monomer(s)

ii) sulfonic-acid-group-containing monomer(s)

iii) nonionic monomer(s)

are preferred according to the invention. By using these terpolymers, it was possible to improve the rinsing performance of machine dishwashing agents according to the invention over comparable dishwashing agents that contain sulfo-polymers without the addition of nonionic monomers.

Preferably, monomers of the general formula R¹(R²)C═C(R³)—X—R⁴, in which R¹ to R³ represent, independently of each other, —H, —CH₃, or —C₂H₅, X represents an optionally present spacer group, which is selected from —CH₂—, —C(O)O—, and —C(O)—NH—, and R⁴ represents a straight-chain or branched saturated alkyl residue having 2 to 22 carbon atoms or an unsaturated, preferably aromatic residue having 6 to 22 carbon atoms, are used as nonionic monomers.

Especially preferred nonionic monomers are butene, isobutene, pentene, 3-methylbutene, 2-methylbutene, cyclopentene, hexene, hexene-1, 2-methylpentene-1, 3-methylpentene-1, cyclohexene, methylcyclopentene, cycloheptene, methylcyclohexene, 2,4,4-trimethylpentene-1, 2,4,4-trimethylpentene-2, 2,3-dimethylhexene-1, 2,4-dimethylhexene-1, 2,5-dimethylhexene-1, 3,5-dimethylhexene-1, 4,4-dimethylhexane-1, ethylcyclohexyn, 1-octene, α-olefins having 10 or more carbon atoms, such as 1-decene, 1-dodecene, 1-hexadecene, 1-octadecene, and C22-α-olefin, 2-styrene, α-methylstyrene, 3-methyl styrene, 4-propylstyrene, 4-cyclohexyl styrene, 4-dodecyl styrene, 2-ethyl-4-benzyl styrene, 1-vinylnaphthalene, 2-vinylnaphthalene, acrylic acid methyl ester, acrylic acid ethyl ester, acryl acid propyl ester, acrylic acid butyl ester, acrylic acid pentyl ester, acrylic acid hexyl ester, methacrylic acid methyl ester, N-(methyl)acrylamide, acrylic acid 2-ethylhexyl ester, methacrylic acid 2-ethylhexyl ester, N-(2-ethylhexyl)acrylamide, acrylic acid octyl ester, methacrylic acid octyl ester, N-(octyl) acrylamide, acrylic acid lauryl ester, methacrylic acid lauryl ester, N-(lauryl) acrylamide, acrylic acid stearyl ester, methacrylic acid stearyl ester, N-(stearyl) acrylamide, acrylic acid behenyl ester, and N-(behenyl) acrylamide, or mixtures thereof.

The percentage of the sulfonic-acid-group-containing copolymers by weight with respect to the total weight of washing or cleaning agents according to the invention is preferably 0.1 to 15 wt %, preferably 1.0 to 12 wt %, and particularly 2.0 to 10 wt %.

The washing or cleaning agents according to the invention can be present in the formulation forms known to a person skilled in the art, i.e., for example in solid or liquid form or as a combination of solid and liquid presentation forms. In particular, powders, granular materials, extrudates, or compacted products, particularly tablets, are suitable as solid presentation forms. The liquid presentation forms based on water and/or organic solvents can be present in a thickened form, in the form of gels.

The washing or cleaning agents according to the invention are preferably present in liquid form. Preferred washing or cleaning agents contain, with respect to the total weight thereof, more than 40%, preferably between 50 and 90 wt %, and particularly between 60 and 80 wt %, of water.

The washing or cleaning agents according to the invention can contain an organic solvent as a further constituent. The addition of organic solvents has an advantageous effect on the enzyme stability and the cleaning performance of these agents. Preferred organic solvents come from the group of mono- or polyhydric alcohols, alkanolamines, or glycol ethers. The solvents are preferably selected from ethanol, n- or i-propanol, butanol, glycol, propane- or butanediol, glycerol, diglycol, propyl- or butyldiglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl, or propyl ether, dipropylene glycol methyl or ethyl ether, methoxy-, ethoxy-, or butoxytriglycol, 1-butoxyethoxy-2-propanol, 3-methyl-3-methoxybutanol, propylene glycol t-butyl ether, and mixtures of these solvents. The percentage of these organic solvents by weight with respect to the total weight of washing or cleaning agents according to the invention is preferably 0.1 to 10 wt %, preferably 0.2 to 8.0 wt %, and particularly 0.5 to 5.0 wt %. Glycerol and 1,2-propylene glycol are especially preferred organic solvents that are especially effective with respect to the stabilization of the washing or cleaning agents. Liquid washing or cleaning agents that contain at least one polyol, preferably from the group of glycerol and 1,2-propylene glycol, wherein the percentage of the polyol by weight with respect to the total weight of the washing or cleaning agent is preferably between 0.1 and 10 wt %, preferably between 0.2 and 8.0 wt %, and particularly between 0.5 and 5.0 wt %, are preferred according to the invention.

Additional preferred organic solvents are the organic amines and alkanolamines. The washing or cleaning agents according to the invention contain these amines preferably in amounts of 0.1 to 10 wt %, preferably 0.2 to 8.0 wt %, and particularly 0.5 to 5.0 wt %, in each case with respect to the total weight of the washing or cleaning agents according to the invention. An especially preferred alkanolamine is ethanolamine.

An additional preferred constituent of the washing or cleaning agents according to the invention is a sugar alcohol (alditol). The group of the alditols comprises non-cyclic polyols of the formula HOCH₂[CH(OH)]ₙCH₂OH. The alditols include, for example, mannite (mannitol), isomalt, lactitol, glucitol (sorbitol), and xylite (xylitol), threitol, erythritol, and arabitol. Sorbitol has proven to be especially advantageous with respect to the enzyme stability. The percentage of the sugar alcohol by weight with respect to the total weight of the washing or cleaning agent is preferably 1.0 to 10 wt %, preferably 2.0 to 8.0 wt %, and particularly 3.0 to 6.0 wt %.

Liquid washing or cleaning agents according to the invention are preferably in multi-phase form, i.e., formulated by combining two or more different liquid washing or cleaning agents, which are separated from each other. This type of formulation increases the stability of the washing or cleaning agent and improves the cleaning performance thereof. A washing or cleaning agent preferred according to the invention is characterized in that it comprises a packaging means and two liquid washing or cleaning agents A and B, which are contained in this packaging means and separated from each other, wherein composition A contains
a) at least one modified protease according to the invention;
b) at least one further enzyme different from the protease according to the invention;
c) 10 to 84.9 wt % of builder(s);
d) 15 to 89.9 wt % of water; and composition B contains
e) 10 to 75 wt % of builder(s);
f) 25 to 90 wt % of water.

### Examples

The following examples illustrate the invention but do not restrict the invention thereto:

### Example 1: Provision of a Protease Having a Poly-Asp-Glu Tag

On the basis of a reference protease that has an amino acid sequence according to SEQ ID NO. 1, a protease variant according to the invention having a C-terminal poly-Asp-Glu elongation was produced by inserting a sequence of codons that codes for alternating Glu and Asp residues, e.g., GAAGAT for Asp-Glu, in a suitable Bacillus production plasmid, by means of which the reference protease can also be produced in good yield, at the 3′ end of the coding region, between the codon for the last amino acid of the protease and the stop codon. After transformation of Bacillus subtilis DB 104, the plasmid was prepared for inspection and the region in question was sequenced. The protease used for the examples below had a C-terminal elongation of (Asp-Glu)₃₂, but proteases having (Asp-Glu)₁₂, (Asp-Glu)₁₆, (Asp-Glu)₂₈, (Asp-Glu)₃₄, (Asp-Glu)₄₆, (Asp-Glu)₆₈, (Asp-Glu)₉₄, (Asp-Glu)₅₀, and (Asp-Glu)₁₀₀ were also produced.

The expression of the protease variant occurred in a manner common in the art by transforming Bacillus subtilis DB 104 (Kawamura and Doi (1984), J. Bacteriol., volume 160 (1), pp. 442-444) by means of a corresponding expression vector and subsequently culturing the transformant that expresses the protease variant. The protease-containing culture supernatants were used for further use, e.g., for example 2.

### Example 2: Production of a Liquid Enzyme Preparation

The supernatant of a cultivation of the expression system, e.g., from example 1, containing the protease according to the invention in an activity of at least 200 HPU/mL, is mixed with 0.1 parts by weight of 1,2-propanediol and stored at 4° C. The protease activity stated in HPU (Henkel protease units) was determined as per van Raay, Saran, and Verbeek, in accordance with the publication “Zur Bestimmung der proteolytischen Aktivität in Enzymkonzentraten und enzymhaltigen Wasch-, Spül- und Reinigungsmitteln” in Tenside (1970), volume 7, pp. 125-132. In the case of storage over several days, the activity is determined again before use.

### Example 3: Mini Washing Test for Determining the Cleaning Performance

The washing performance of the protease to be tested is examined in comparison with a known reference protease, which was produced in parallel under the same conditions. For this purpose, an (enzyme-free) liquid washing agent formulation (see example 5, prepared without enzyme for this test) is dissolved in waters of different degrees of hardness (0-56 dGH) in a typical concentration (78 g/17 L) and mixed with amounts of the test protease and reference protease of equal activity (10 HPU/mL). In the miniaturized washing test (1 mL of washing liquid, 48-well plate with lid, stain of Ø 1 cm), standardized commercially available stains (EMPA164, CFT PC10, CTF 10N, CFT C03, EMPA122, CFT C05) on (polyester) cotton woven fabric are incubated in this washing liquid for 60 minutes at 40° C. with pivoting. After rinsing (tap water), drying, and fixing of the stains, they are measured with a spectrophotometer (Konica Minolta Cm700d) and the brightness differences ΔL* between each sample and a zero value of corresponding water hardness also washed analogously without enzyme are summed over all 6 stains and evaluated as a measure of the washing performance.

### Example 4: Evaluation of the Water Hardness Tolerance

The water hardness tolerance of the example protease from example 2 is determined in comparison to the reference protease in the mini washing test (example 3). The reference protease is the reference protease mentioned in example 1.

For the example protease, the washing performance is determined in accordance with example 3 at the water hardnesses indicated in the following table. The sum of the brightness differences (i.e., the washing performance) at a water hardness of 0 dGH is set to 100% and the washing performance at the higher water hardnesses is set in relation thereto in percent.

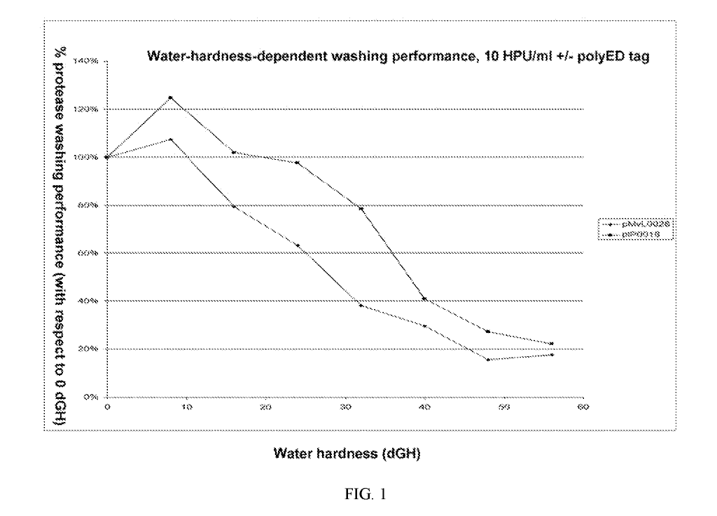

From the table and FIG. 1, it is clear that the decrease in the washing performance of the example protease according to the invention with increasing water hardness is weaker than in the case of the reference protease and thus its water hardness tolerance is greater.

| | Comparison enzyme | Example enzyme having |
|---|---|---|
| | without extension | C-terminal poly-Asp-Glu extension |
| | (SEQ ID NO. 1) | (Asp-Glu)₃₂ |
| dGH | % (with respect to 0 dGH) | % (with respect to 0 dGH) |
| 0 | 100% | 100% |
| 8 | 108% | 125% |
| 16 | 79% | 102% |
| 24 | 63% | 98% |
| 32 | 38% | 78% |
| 40 | 30% | 41% |
| 48 | 16% | 27% |
| 56 | 18% | 22% |

### Example 5: Washing Agent Formulation Having Hardness-Tolerant Protease

The composition of some preferred washing or cleaning agents can be found in the following tables (specifications in wt % with respect to the total weight of the washing or cleaning agent, unless otherwise indicated).

| | Formula 1 | Formula 2 | Formula 3 | Formula 4 | Formula 5 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Enzyme ** | 0.0005 to 1.0 | 0.001 to 0.5 | 0.002 to 0.2 | 0.004 | 0.012 |
| Misc. | ad 100 | ad 100 | ad 100 | ad 100 | ad 100 |

** enzyme different from the protease according to the invention

| | Formula 6 | Formula 7 | Formula 8 | Formula 9 | Formula 10 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Misc. | ad 100 | ad 100 | ad 100 | ad 100 | ad 100 |

| | Formula 11 | Formula 12 | Formula 13 | Formula 14 | Formula 15 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Water | >40 | 50 to 85 | 60 to 80 | 64 | 71 |
| Misc. | ad 100 | ad 100 | ad 100 | ad 100 | ad 100 |

| | Formula 16 | Formula 17 | Formula 18 | Formula 19 | Formula 20 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Builder | 5.0 to 40 | 10 to 30 | 12 to 25 | 26 | 18 |
| Water | >40 | 50 to 85 | 60 to 80 | 64 | 71 |
| Misc. | ad 100 | ad 100 | ad 100 | ad 100 | ad 100 |

| | Formula 21 | Formula 22 | Formula 23 | Formula 24 | Formula 25 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Nonionic | 0.2 to 10 | 0.4 to 7.0 | 0.6 to 6.0 | 4.0 | 2.0 |
| surfactant | | | | | |
| Water | >40 | 50 to 85 | 60 to 80 | 64 | 71 |
| Misc. | ad 100 | ad 100 | ad 100 | ad 100 | ad 100 |

| | Formula 26 | Formula 27 | Formula 28 | Formula 29 | Formula 30 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Builder | 5.0 to 40 | 10 to 30 | 12 to 25 | 26 | 18 |
| Nonionic | 0.2 to 10 | 0.4 to 7.0 | 0.6 to 6.0 | 4.0 | 2.0 |
| surfactant | | | | | |
| Water | >40 | 50 to 85 | 60 to 80 | 64 | 71 |
| Misc. | ad 100 | ad 100 | ad 100 | ad 100 | ad 100 |

| | Formula 31 | Formula 32 | Formula 33 | Formula 34 | Formula 35 |
|---|---|---|---|---|---|
| Modified protease | 0.005 to 1.0 | 0.01 to 0.5 | 0.02 to 0.2 | 0.06 | 0.17 |
| according to the | | | | | |
| invention | | | | | |
| Amylase | 0.0005 to 1.0 | 0.001 to 0.5 | 0.002 to 0.2 | 0.004 | 0.012 |
| Penta-potassium | 5.0 to 40 | 10 to 30 | 12 to 25 | 18 | 12 |
| triphosphate | | | | | |
| HEDP | 0.1 to 8.0 | 0.2 to 5.0 | 0.5 to 3.0 | 3.0 | 2.0 |
| Sulfo-copolymer | 0.1 to 15 | 1.0 to 12 | 2.0 to 10 | 4.0 | 6.0 |
| Hydroxy mixed | 0.2 to 10 | 0.4 to 7.0 | 0.6 to 6.0 | 4.0 | 2.0 |
| ether | | | | | |
| Water | >40 | 50 to 85 | 60 to 80 | 64 | 71 |
| Misc. | ad 100 | ad 100 | ad 100 | ad 100 | ad 100 |

The present invention also relates to the use of a modified protease according to the invention to improve the water hardness tolerance of washing or cleaning agents.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A protease-polyanion fusion protein, containing
a) a protease comprising an amino acid sequence that is at least 80% identical to one of the sequences indicated in SEQ ID NO. 1 to SEQ ID NO. 5 over the entire length of said one of the amino acid sequences indicated in SEQ ID NO. 1 to SEQ ID NO. 5; and
b) a polyanion.

2. The protease-polyanion fusion protein according to claim 1, wherein the polyanion has a length of 10 to 1000.

3. The protease-polyanion fusion protein according to claim 1, wherein the polyanion is constructed completely or predominantly of aspartate and/or glutamate residues.

4. The protease-polyanion fusion protein according to claim 1, wherein the polyanion is bonded C-terminally to the protease.

5. A nucleic acid that codes for a protease-polyanion fusion protein according to claim 1.

6. A vector containing a nucleic acid according to claim 5.

7. A non-human host cell that contains a nucleic acid according to claim 5 wherein the host cell secretes the protease-polyanion fusion protein into medium surrounding the host cell.

8. A method for producing a protease-polyanion fusion protein comprising:
a) cultivating a host cell according to claim 7; and
b) isolating the protease from the medium or from the host cell.

9. An agent, wherein the agent contains at least one protease-polyanion fusion protein according to claim 1.

10. A method for improving the water hardness tolerance of washing or cleaning agents utilizing the protease-polyanion fusion protein according to claim 1.

11. A protease-polyanion fusion protein, containing
a) a protease comprising an amino acid sequence that is at least 80% identical to one of the sequences indicated in SEQ ID NO. 1 to SEQ ID NO. 5 over the entire length of said one of the amino acid sequences indicated in SEQ ID NO. 1 to SEQ ID NO. 5; and
b) a polyanion that is constructed completely or predominantly of aspartate and/or glutamate residues.

12. A protease-polyanion fusion protein, containing
a) a protease comprising an amino acid sequence that is at least 80% identical to one of the sequences indicated in SEQ ID NO. 1 to SEQ ID NO. 5 over the entire length of said one of the amino acid sequences indicated in SEQ ID NO. 1 to SEQ ID NO. 5; and
b) a polyanion that is bonded C-terminally to the protease.

13. The protease-polyanion fusion protein according to claim 2, wherein the polyanion is constructed completely or predominantly of aspartate and/or glutamate residues.

14. The protease-polyanion fusion protein according to claim 2, wherein the polyanion is bonded C-terminally to the protease.

15. The protease-polyanion fusion protein according to claim 3, wherein the polyanion is bonded C-terminally to the protease.

16. A non-human host cell that contains a protease-polyanion fusion protein according to claim 1.

17. A non-human host cell that contains a nucleic acid according to claim 5.

18. A non-human host cell that contains a vector according to claim 6.

19. The vector according to claim 6 further defined as a cloning vector or expression vector.

20. The agent according to claim 9 further defined as a washing or cleaning agent.
